(19) United States
(12) Patent Application Publication
(10) Pub. No.: US 2022/0139526 A1
(43) Pub. Date: May 5, 2022

(54) SYSTEMS AND METHODS TO PREPARE DICOM FILES FOR CLOUD STORAGE

(71) Applicant: Medical Insights Diagnostic Centers, Inc., Palm Desert, CA (US)

(72) Inventor: Virgil L. Williams, Palm Desert, CA (US)

(21) Appl. No.: 17/089,569

(22) Filed: Nov. 4, 2020

(51) Int. Cl. G16H 30/20; G16H 10/20; G16H 50/70; G16H 30/40; G06F 16/51; G06F 16/55

(52) U.S. Cl. CPC G16H 30/20; G16H 10/20; G06F 16/55; G16H 30/40; G06F 16/51; G16H 50/70

(57) ABSTRACT

Systems and methods to prepare DICOM files for cloud storage are disclosed. Exemplary implementations may: extract a DICOMDIR file from a disk; parse the values to the key attributes to identify at least the values for the study instance UID and the values for the study date; group the DICOM files by the values of the study instance UID; for every group comprising the studies list, generate, based on the parse, a study name value and a viewer URL parameter; and for every group comprising the studies list, generate a separate DICOMDIR file so that the separate DIRCOMDIR file includes the group of the DICOM files that are associated with a study represented by the study instance UID.

### FIELD OF THE DISCLOSURE

The present disclosure relates to systems and methods to prepare DICOM files for cloud storage.

### BACKGROUND

Sharing medical information (e.g., medical images, medical records, documents, reports, results, images) with medical professionals, consultants, and/or specialists in a timely manner is imperative for accurate diagnosis and treatment for a patient. Sharing the medical information between the medical professionals may place a burden on them to sort and/or organize the medical information received. Additionally, the medical information must be kept in a HIPAA compliant system whether stored electronically or in hard copy. In particular, medical images are store in a Digital Imaging and Communications in Medicine (DICOM) format of which are stored and accessible via a Picture Archiving and Communication System (PACS). If caregivers do not have access to a PACS, the medical images may be viewed from disks. In some healthcare facilities, the disks may be obsolete to create and/or view and may be misplaced.

### SUMMARY

One aspect of the present disclosure related to a system configured to prepare DICOM files for cloud-based storage upon possession of a disk with medical images in the DICOM format. The disk may include a Digital Imaging and Communications in Medicine directory (DICOMDIR) file of which includes key attributes related to studies, patients, series, and images on the disk and paths to the DICOM files. The DICOMDIR file may be extracted for subsequent parsing of the key attributes. The parsing of the key attributes may identify distinct studies. Thus, for individual studies, the images related to a given study may be grouped. A study name and viewer URL parameter may be generated for the groups of images to enable searchability of the studies and its images by users. Lastly, a separate DICOMDIR file may be generated for the individual groups so that the DICOMDIR includes the images associated with a given study. Therefore, the DICOMDIR files may be imported by a cloud-based PACS for storage and remote accessibility by users. As used herein, the terms “DICOM files”, DICOM file images, “DICOM images”, “images”, “medical images” or variations thereof may be used interchangeably and refer to a medical image in the DICOM format.

One aspect of the present disclosure relates to a system configured to prepare DICOM files for cloud storage. The system may include one or more hardware processors configured by machine-readable instructions. Machine-readable instructions may include one or more instruction components. The instruction components may include computer program components. The instruction components may include one or more of directory management component, value parsing component, study organization component, user input component, and/or other instruction components.

The directory management component may be configured to extract a DICOMDIR file from a disk. The DICOMDIR file may include values to key attributes of individual DICOM files and/or other information. The key attributes may include a study instance unique identifier (UID), a study date, and/or other attributes. Individual studies may be defined by a value to the study instance UID, a value to the study date, and/or other values.

The value parsing component may be configured to parse the values to the key attributes to identify at least the values for the study instance unique identifier, the values for the study date, and/or other information. The values to the study instance UID may indicate how many studies are included in the DICOMDIR file.

The study organization component may be configured to group the DICOM files by the values of the study instance UID. Groups of DICOM files may comprise a studies list. For every group including the studies list, the study organization component may be configured to generate a study name value and a viewer URL parameter. Generation may be based on the parse.

For every group including the studies list, the directory management component may be configured to generate a separate DICOMDIR file. As such, the separate DICOMDIR file may include the group of the DICOM files that are associated with a given study represented by the study instance UID.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof. j

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to prepare DICOM files for cloud storage, in accordance with one or more implementations.

FIG. 2 illustrates a method to prepare DICOM files for cloud storage, in accordance with one or more implementations.

FIG. 3 illustrates an example implementation of the system configured to prepare DICOM files for cloud storage, in accordance with one or more implementations.

### DETAILED DESCRIPTION

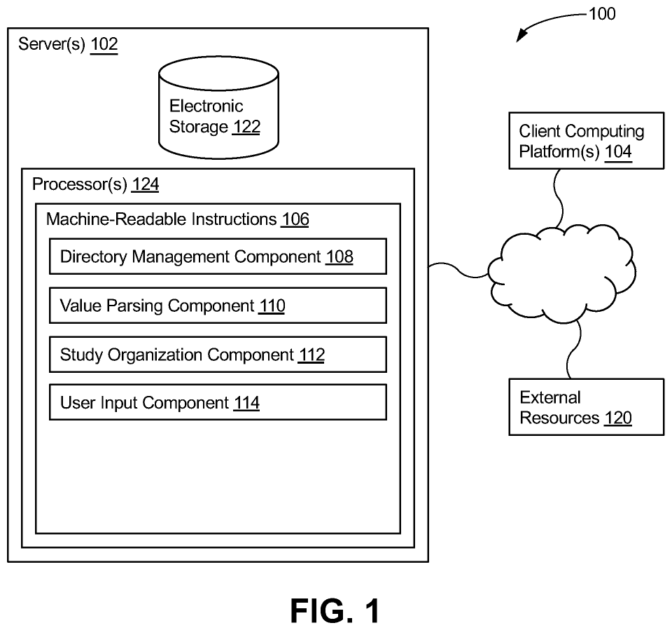

FIG. 1 illustrates a system 100 configured to prepare DICOM files for cloud storage, in accordance with one or more implementations. In some implementations, system 100 may include one or more servers 102. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Server(s) 102 may be configured by machine-readable instructions 106. Machine-readable instructions 106 may include one or more instruction components. The instruction components may include computer program components. The instruction components may include one or more of directory management component 108, value parsing component 110, study organization component 112, user input component 114, and/or other instruction components.

Directory management component 108 may be configured to extract a DICOMDIR file from a disk. By way of non-limiting example, the disk may include a DVD, a CD, and/or other disk. The DICOMDIR file may include values to attributes of individual DICOM files, paths to the DICOM files, and/or other information. Attributes for the individual DICOM files may include a study instance UID, a study date, a patient identifier, a patient name, a study ID, a referring physician's name, a patient birthdate, series number, modality, pixel data, and other attributes. A study instance UID value may be unique to individual studies to distinguish a given study from other studies. A study date value may define a date of which the study was conducted. A patient identifier value may identify a patient. A patient name value may define the name (e.g., first name, last name, middle name, suffix) of the patient. In some implementations, the patient identifier value may be a medical record number associated with a healthcare system. A study ID value may be an identifier generated by a user (e.g., medical professional) or by equipment (e.g., an MRI machine). A referring physical's name value may define a doctor, a physician, a specialist, and/or other medical professional that the patient was referred by for a medical image. A patient birthdate value may define the date of birth of the patient. A series number value may identify a series among other series. A modality value may define a type of equipment (e.g., MR, CT) used to create the image. A value for the pixel data may define a medical image in accordance with values to one or more other attributes that define the DICOM files (e.g., pixel spacing, pixel representation among others). A medical image may include an X-ray, a computed tomography (CT) scan , a magnetic resonance imaging (MRI), ultrasound, an electrocardiogram (EKG), medical illustrations, video recordings, photographs (e.g., of a hazardous work environment, an accident scene), lab results, visual aids, and/or other medical images. Key attributes of the individual DICOM files may include the study instance UID, the study date the patient identifier, the patient name, the study ID, and/or other key attributes. Individual studies may be defined by a value to the study instance UID and the value to the study date. By way of non-limiting illustration, a first study may be defined by a first value (e.g., 1122) to the study instance UID and a second study may be defined by a second value (e.g., 3344) to the study instance UID that is different from the first value.

Value parsing component 110 may be configured to parse the values to the key attributes and/or the values to other ones of the attributes. Parsing the values to the key attributes may be to identify at least the values for the study instance UID, the values for the study date, a study description, and/or other attributes. The values to the study instance UID may indicate how many studies are included in the DICOMDIR file due to how may values to the study instance UID are identified. The study description may describe various details of the study or part thereof. In some implementations, value parsing component 110 may be configured to parse the values to the attributes that define the individual DICOM files to identify values to various attributes such as the referring physician's name, the modality, a description, a retrieval application entity (AE) title, and/or other dates (e.g., content date), other titles, and/or other information. The values for the description may describe a strain, a container, or otherwise the define a given study or part thereof. The application entity may be a local or remote DICOM service that may store, query, and/or other actions related to DICOM files. The values for the retrieval AE title may be a title for an application entity where a particular instance may be obtained from a network.

Study organization component 112 may be configured to group the DICOM files by the values of the study instance UID. Groups of DICOM files may comprise a studies list. Thus, the studies list includes unique studies conducted and included in the DICOMDIR file. The individual DICOM files are defined by a value to the study instance UID among other attribute values. The study instance UID value defining a given DICOM file indicates which study the DICOM file is associated with. As such, grouping the DICOM files by the values of the study instance UID includes parsing the attributes of each DICOM file to identify the the study instance UID value and grouping the DICOM files with the same study instance UID value together as one study. By way of non-limiting illustration, a first DICOM file and a second DICOM file may both be defined by the first value (e.g., 1122) to the study instance UID and a third DICOM file and a fourth DICOM file may both be defined by the second value (e.g., 3344) to the study instance UID. Thus, the first DICOM file and the second DICOM file are part of the first study and the third DICOM file and the fourth DICOM file are part of the second study.

For every group comprising the studies list, study organization component 112 may be configured to generate a study name value, a viewer URL parameter, and/or other parameters or values. The generation may be based on the parse. The study name value may define name by which the associated study (i.e., study instance UID) may be searchable by as opposed to a sequence of numbers that is the study instance UID. In some implementations, generating the study name value may be based on values to the study ID, the patient name, and/or other attributes. In some implementations, the study name value may be based on user input from a user, modified based on the user input, automatically generated by study organization component 112, or generated by way of another method.

In some implementations, user input component 114 may be configured to receive user input. In some implementations, the user input may include a value to the viewer URL parameter. The user input may be entered and subsequently received by user input component 114 by one or more user interface elements. User interface elements may be configured to facilitate user interaction with a user interface of client computing platform 104, user entry via the user interface, and/or selection via the user interface. By way of non-limiting illustration, the user interface elements may include one or more of text input fields, drop down menus, check boxes, display windows, virtual buttons, and/or other user interface elements. In some implementations, the user input may indicate initiating the extraction of the DICOMDIR file. The user input indicating the initiation of the extraction may be via the one or more user interface elements. For example, individual user interface element (e.g., a virtual button) may be configured to be selected by the user to cause the extraction. As a result, the extraction of the DICOMDIR file may be responsive to receipt of the user input indicating the initiation of the extraction.

For every group comprising the studies list, directory management component 108 may be configured to generate a separate DICOMDIR file. As such, the separate DICOMDIR file includes the group of the DICOM files that are associated with a given study represented by the study instance UID value. Thus, the individual DICOMDIR files generated may store paths to the DICOM files of the given study. By way of non-limiting illustration, a first DICOMDIR file and a second DICOMRDIR file may be generated. The first DICOMDIR file may be associated with the DICOM files of the first study and thus store paths to the first DICOM file and the second DICOM file. Likewise, second DICOMDIR file may be associated with the DICOM files of the second study and thus store paths to the third DICOM file and the fourth DICOM file.

In some implementations, directory management component 108 may be configured to store the generated DICOMDIR files to individual datastores stored in datasets that are associated with the individual patients. An individual patient may be associated with an individual dataset of which stores information related to the patient, studies of the patient, and/or other information. The individual datasets may store the individual datastores that are specifically compatible with DICOM files. The individual datastores may store the DICOM files related to a particular patient. Thus, the first DICOMDIR file generated for the first study may be stored to a datastore associated with a first patient of which the first study was conducted for.

The separate DICOMDIR files, the datastores, the datasets, and/or other information may be prepared by system 100 for importing for one or more cloud-based PACS. The cloud-based PACSs may provide storage and acquisition of the medical images by cloud-based servers so that the medical images are accessible from anywhere without burdens related to hardware and software often put up organizations in possession of a PACS. Upon the separate DICOMDIR files, the datastores, the datasets, and/or other information being imported by the one or more cloud-based PACS, studies may be more easily searchable and thus accessible by users to acquire the medical images for a given study.

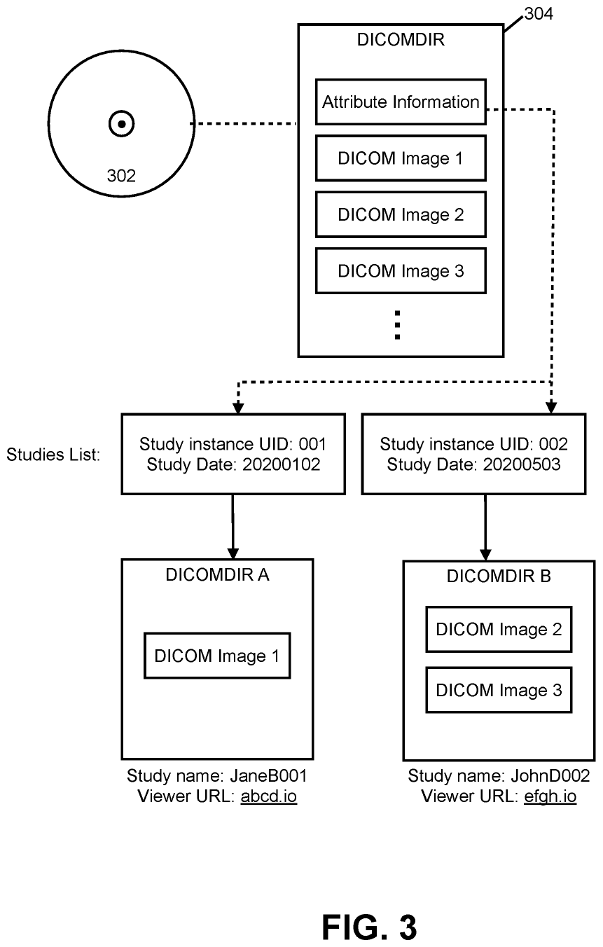

FIG. 3 may illustrate an example implementation of the system configured to prepare DICOM files for cloud storage, in accordance with one or more implementations. FIG. 3 may illustrate a disk 302. Disc 302 may include a DICOMDIR file 304 that is extracted from disk 302. The DICOMDIR file 304 may include attribute information, and one or more DICOM image files (e.g., DICOM Image 1, DICOM Image 2, and DICOM Image 3). The attribute information may include key attributes and values to the key attributes. The values to the key attributes may be parsed to identify distinct values to study instance UIDs, 001 and 002, and study dates, 20200102 and 20200503, respectively. Thus, it may be determined that the DICOMDIR file includes two studies. By parsing the attribute values of the DICOM image files to identify the study instance UID that each DICOM image file is defined by, the DICOM image files may be grouped by the same study instance UID (i.e., the same study). Thus, DICOM Image 1 may be associated with study instance UID 001 and DICOM Image 2 and DICOM Image 3 may be associated with study instance 002 and thus grouped together. Subsequently, new DICOMDIR files, DICOMDIR A and DICOMDIR B, may be generated for each one of the groups (i.e., each study) to store paths to the DICOM image files associated with each study instance UID. For each DICOMDIR A and DICOMDIR B, a study name value and a viewer URL parameter may be generated, JaneB001 and JohnD002, respectively. Viewer URLs, abcd.io and efgh.io, may be input by a user as the accessibility to the DICOMDIR files and thus the DICOM Images. As such, DICOM image files may be imported by virtual PACS, searchable by the study name values, and accessible via a value to the viewer URL parameter.

In some implementations, server(s) 102, client computing platform(s) 104, and/or external resources 120 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 120 may be operatively linked via some other communication media.

A given client computing platform 104 may include one or more processors configured to execute computer program components. The computer program components may be configured to enable an expert or user associated with the given client computing platform 104 to interface with system 100 and/or external resources 120, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 120 may include sources of information outside of system 100, external entities participating with system 100, and/or other resources. In some implementations, some or all of the functionality attributed herein to external resources 120 may be provided by resources included in system 100.

Server(s) 102 may include electronic storage 122, one or more processors 124, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 122 may comprise non-transitory storage media that electronically stores information. The electronic storage media of electronic storage 122 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 122 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 122 may include one or more virtual storage resources (e.g., cloud storage, a virtual private network, and/or other virtual storage resources). Electronic storage 122 may store software algorithms, information determined by processor(s) 124, information received from server(s) 102, information received from client computing platform(s) 104, and/or other information that enables server(s) 102 to function as described herein.

Processor(s) 124 may be configured to provide information processing capabilities in server(s) 102. As such, processor(s) 124 may include one or more of a digital processor, an analog processor, a digital circuit designed to process information, an analog circuit designed to process information, a state machine, and/or other mechanisms for electronically processing information. Although processor(s) 124 is shown in FIG. 1 as a single entity, this is for illustrative purposes only. In some implementations, processor(s) 124 may include a plurality of processing units. These processing units may be physically located within the same device, or processor(s) 124 may represent processing functionality of a plurality of devices operating in coordination. Processor(s) 124 may be configured to execute components 108, 110, 112, and/or 114, and/or other components. Processor(s) 124 may be configured to execute components 108, 110, 112, and/or 114, and/or other components by software; hardware; firmware; some combination of software, hardware, and/or firmware; and/or other mechanisms for configuring processing capabilities on processor(s) 124. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, and/or 114 are illustrated in FIG. 1 as being implemented within a single processing unit, in implementations in which processor(s) 124 includes multiple processing units, one or more of components 108, 110, 112, and/or 114 may be implemented remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other ones of components 108, 110, 112, and/or 114. As another example, processor(s) 124 may be configured to execute one or more additional components that may perform some or all of the functionality attributed below to one of components 108, 110, 112, and/or 114.

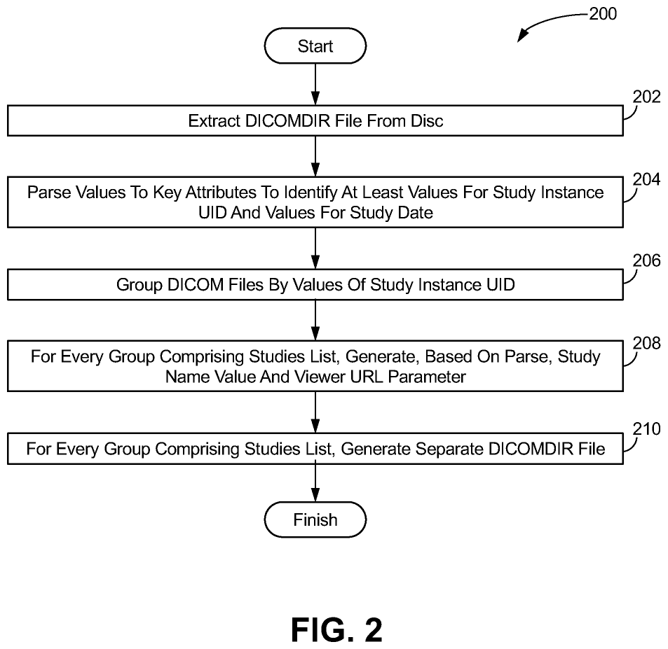

FIG. 2 illustrates a method 200 to prepare DICOM files for cloud storage, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include extracting a DICOMDIR file from a disk. The DICOMDIR file may include values to key attributes of individual ones of the DICOM files. The key attributes may include a study instance unique identifier and a study date. Individual studies may be defined by a value to the study instance UID and a value to the study date. Operation 202 may be performed by one or more hardware processors configured by machine-readable instructions including a component that is the same as or similar to directory management component 108, in accordance with one or more implementations.

An operation 204 may include parsing the values to the key attributes to identify at least the values for the study instance UID and the values for the study date. The values to the study instance UID may indicate how many studies are included in the DICOMDIR file. Operation 204 may be performed by one or more hardware processors configured by machine-readable instructions including a component that is the same as or similar to value parsing component 110, in accordance with one or more implementations.

An operation 206 may include grouping the DICOM files by the values of the study instance UID. Groups of DICOM files may comprise a studies list. Operation 206 may be performed by one or more hardware processors configured by machine-readable instructions including a component that is the same as or similar to study organization component 112, in accordance with one or more implementations.

An operation 208 may include generating, based on the parse, a study name value and a viewer URL parameter. The study name value and the viewer URL parameter may be generated for every group comprising the studies list. Operation 208 may be performed by one or more hardware processors configured by machine-readable instructions including a component that is the same as or similar to study organization component 112, in accordance with one or more implementations.

An operation 210 may include generating a separate DICOMDIR file for every group comprising the studies list. Thus, the separate DICOMDIR file may include the group of the DICOM files that are associated with a study represented by the study instance UID. Operation 210 may be performed by one or more hardware processors configured by machine-readable instructions including a component that is the same as or similar to directory management component 108, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured to prepare DICOM files for cloud storage, the system comprising:
one or more processors configured by machine-readable instructions to:
extract a DICOMDIR file from a disk, wherein the DICOMDIR file comprises of values to key attributes of individual DICOM files, wherein the key attributes include a study instance unique identifier (UID) and a study date, wherein individual studies are defined by a value to the study instance UID and a value to the study date;
parse the values to the key attributes to identify at least the values for the study instance UID and the values for the study date, wherein the values to the study instance UID indicate how many studies are included in the DICOMDIR file;
group the DICOM files by the values of the study instance UID, wherein groups of DICOM files comprise a studies list;
for every group comprising the studies list, generate, based on the parse, a study name parameter, a value to the study name parameter, and a viewer URL parameter; and
for every group comprising the studies list, generate a separate DICOMDIR file so that the separate DICOMDIR file includes the group of the DICOM files that are associated with a study represented by the study instance UID.

2. The system of claim 1, wherein the key attributes include a patient identifier, a patient name, and a study ID.

3. The system of claim 2, wherein attributes for the individual DICOM files include a referring physician's name, a patient birthdate, series number, modality, and the key attributes.

4. The system of claim 2, wherein generating the study name value is based on values to the study ID and/or the patient name.

5. The system of claim 1, wherein the one or more processors are further configured by the machine-readable instructions to:
receive user input, wherein the user input indicates initiating the extraction of the DICOMDIR file, wherein the extraction of the DICOMDIR file is responsive to receipt of the user input.

6. The system of claim 1, wherein the DICOMDIR file comprises paths to the DICOM files.

7. The system of claim 5, wherein the user input includes a value to the viewer URL parameter.

8. A method to prepare DICOM files for cloud storage, the method comprising:
extracting a DICOMDIR file from a disk, wherein the DICOMDIR file comprises of values to key attributes of individual DICOM files, wherein the key attributes include a study instance unique identifier (UID) and a study date, wherein individual studies are defined by a value to the study instance UID and a value to the study date;
parsing the values to the key attributes to identify at least the values for the study instance UID and the values for the study date, wherein the values to the study instance UID indicate how many studies are included in the DICOMDIR file;
grouping the DICOM files by the values of the study instance UID, wherein groups of DICOM files comprise a studies list;
for every group comprising the studies list, generating, based on the parse, a study name parameter, a value to the study name parameter, and a viewer URL parameter; and
for every group comprising the studies list, generating a separate DICOMDIR file so that the separate DICOMDIR file includes the group of the DICOM files that are associated with a study represented by the study instance UID.

9. The method of claim 8, wherein the key attributes include a patient identifier, a patient name, and a study ID.

10. The method of claim 9, wherein attributes for the individual DICOM files include a referring physician's name, a patient birthdate, series number, modality, and the key attributes.

11. The method of claim 9, wherein generating the study name value is based on values to the study ID and/or the patient name.

12. The method of claim 8, further comprising:
receiving user input, wherein the user input indicates initiating the extraction of the DICOMDIR file, wherein the extraction of the DICOMDIR file is responsive to receipt of the user input.

13. The method of claim 8, wherein the DICOMDIR file comprises paths to the DICOM files.

14. The method of claim 12, wherein the user input includes a value to the viewer URL parameter.
